(19) United States
(12) Patent Application Publication
(10) Pub. No.: US 2025/0114523 A1
(43) Pub. Date: Apr. 10, 2025

(54) AN IMPLANTABLE CLOSED-LOOP SYSTEM FOR MONITORING AND TREATMENT

(71) Applicant: Massachusetts Institute of Technology, Cambridge, MA (US)

(72) Inventors: Anantha Chandrakasan, Belmont, MA (US); Carlo Giovanni Traverso, Newton, MA (US); Hen Wei Huang, Watertown, MA (US); SeungHo Lee, Arlington, MA (US); Saurav Maji, Cambridge, MA (US); Peter Chai, Waban, MA (US); Tom Kerssemakers, Amsterdam (NL); Ian Ballinger, Watertown, MA (US)

(73) Assignees: Massachusetts Institute of Technology, Cambridge, MA (US); The Brigham and Women's Hospital, Inc., Boston, MA (US)

(21) Appl. No.: 18/727,623

(22) PCT Filed: Nov. 23, 2022
(86) PCT No.: PCT/US2022/080385

§ 371 Date: Jul. 9, 2024

Related U.S. Application Data
(60) Provisional application No. 63/303,229, filed on Jan. 26, 2022.

(51) Int. Cl. A61M 5/172; A61M 5/142

(52) U.S. Cl. CPC A61M 5/1723; A61M 5/14216; A61M 5/14276; A61M 2205/3317; A61M 2205/3327; A61M 2205/3375; A61M 2205/8237; A61M 2205/8243; A61M 2230/04; A61M 2230/06; A61M 2230/42; A61M 2230/50

(57) ABSTRACT

A device configured to be implanted in a subject includes a sensing module, a therapeutic module, and a battery. The sensing module includes at least two of an accelerometer, an electrocardiogram (ECG) sensor, a photoplethysmogram (PPG) sensor, and a temperature sensor. The therapeutic module includes a drug reservoir and a reciprocating pump. The battery powers the sensing module and the therapeutic module. The sensing module is configured to detect a biological event in the subject and, upon detection of the biological event, send a signal to the therapeutic module. The therapeutic module is configured to receive the signal from the sensing module and, upon receiving the signal, administer a drug to the subject from the drug reservoir via the pump.

### CROSS-REFEENCE TO RELATED APPLICATION(S)

This application claims the priority benefit, under 35 U.S.C. 119(e), of U.S. Application No. 63/303,229, filed Jan. 26, 2022, which is incorporated herein by reference in its entirety.

### BACKGROUND

Doctors and nurses continuously monitor patient vital signs in hospital. For example, patient vital sign monitoring is common in the intensive-care unit (ICU), the operating theatre, and the post-surgery waiting room of the hospital. Vital sign monitoring can help hospital staff provide timely treatment and mitigate morbidity to achieve better clinical outcomes.

Outside of the hospital setting, continuous monitoring of vital signs is much less common. Systems for monitoring patients at home usually include bulky, costly, and immobile equipment. Because the equipment is immobile, patients using these systems are unable to move about freely or leave their homes. These systems are typically only used with the sickest patients.

If a patient's at-home vital sign monitoring system detects abnormal vital signs, the system may send alarms to the patient, to the patient's emergency contact, and/or to a healthcare professional associated with the patient. One of these individuals may then review the patient's vital sign data and administer treatment if necessary. However, there are downsides to this approach. A patient experiencing a medical emergency may not be able to self-administer treatment. Furthermore, the patient's emergency contact or healthcare professional may not be readily available to provide timely treatment.

### SUMMARY

In one embodiment, a device is configured to be implanted in a subject. The device includes a sensing module comprising at least two of an accelerometer; an electrocardiogram (ECG) sensor; a photoplethysmogram (PPG) sensor; and a temperature sensor. The device also includes a therapeutic module coupled to the sensing module. The therapeutic module includes a drug reservoir and a reciprocating pump. The device also includes a battery coupled to the sensing module and the therapeutic module. The battery is configured to provide power to the sensing module and the therapeutic module. The sensing module is configured to detect a biological event in the subject and, upon detection of the biological event, send a signal to the therapeutic module. The therapeutic module is configured to receive the signal from the sensing module and, upon receiving the signal, administer a drug to the subject from the drug reservoir via the reciprocating pump.

In another embodiment, a method of a method of using a device includes measuring the subject's respiratory rate, heart rate, blood oxygen saturation, and temperature with the sensing module; while measuring, detecting a biological event in the subject; and upon detecting the biological event, actuating a pump to administer the drug from the drug reservoir to the subject. The device includes a sensing module; a therapeutic module including a drug reservoir containing a drug and a pump; and a rechargeable battery configured to provide power to the device; wherein the device is fully implanted in a subject.

In another embodiment, a system is configured to be implanted in a subject. The system includes a housing including a cavity and a sensing module disposed in the cavity. The sensing module includes at least two of a respiratory rate sensor; a heart rate sensor; a blood oxygen saturation sensor; and a temperature sensor. The system further includes a reciprocating pump coupled to the sensing module and disposed at least partially in the cavity. The reciprocating pump is configured to administer a medicine to the subject. The system further includes a battery disposed in the cavity and coupled to the sensing module and the pump. The battery is configured to provide power to the sensing module and the reciprocating pump.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1 shows an embodiment of an implantable device.

FIG. 2A shows an exploded view of another embodiment of an implantable device.

FIG. 2B shows another view of the implantable device shown in FIG. 2A.

FIG. 2C shows an exploded view of the therapeutic module in the implantable device shown in FIG. 2A.

FIG. 3 shows an embodiment of a therapeutic module.

FIG. 4 shows a reciprocating pumping mechanism in the therapeutic module in FIG. 3.

FIG. 5A shows current draw vs. time from a motor driving a reciprocating pump in an implantable device using various voltages.

FIG. 5B shows voltage vs. capacity from a motor driving a reciprocating pump in an implantable device using various currents.

FIG. 6 shows current draw vs. time from a motor driving a reciprocating pump in an implantable device using a current limiter.

FIG. 7A shows circuit diagram of a voltage regulator for a programmable soft starter.

FIG. 7B shows output voltage vs. time of the regulator shown in FIG. 7A.

FIG. 7C shows current draw vs. time of the regulator shown in FIG. 7A for various capacitances.

FIG. 7D shows a circuit diagram of components used for a pulse-width modulated soft start strategy for a motor driving a reciprocating pump in an implantable device.

FIG. 7E shows inrush current vs. time of the motor in FIG. 7D.

FIG. 7F shows a circuit diagram of components used for another pulse-width modulated soft start strategy for motor driving a reciprocating pump in an implantable device.

FIG. 7G shows various current and voltage draw from the motor in FIG. 7F.

FIG. 8 is a circuit diagram of a power management architecture.

FIG. 9 shows current vs. time for a non-linear soft start of a motor with and without a loading.

FIG. 10A shows battery voltage vs. capacity during pulsed operation of the pump motor.

FIG. 10B shows voltage vs. number of cycles for pulsing a pump motor on and off using various soft start strategies.

FIG. 10C shows pumped out volume vs. time with various driving voltages.

FIG. 11 is a diagram of the closed-loop controller for an implantable device.

FIG. 12 shows implant locations for an implantable device.

FIG. 13 shows ECG signals measured in various locations and orientations in the body.

FIG. 14A is a circuit diagram of the controller for an implantable device.

FIG. 14B is a circuit diagram of an inertial measurement unit (IMU) in an implantable device.

FIG. 14C is a circuit diagram of an electrocardiogram (ECG) sensor in an implantable device.

FIG. 14D is a circuit diagram of a photoplethysmogram (PPG) sensor in an implantable device.

FIG. 14E is a circuit diagram of a temperature sensor in an implantable device.

FIG. 14F is a circuit diagram of a motor for a reciprocating pump in an implantable device.

FIG. 14G is a circuit diagram of a wireless charging unit in an implantable device.

FIG. 14H is a circuit diagram of connectors in an implantable device.

FIG. 14I is a circuit diagram of a voltage regulator in an implantable device.

FIG. 14J is a circuit diagram of an inter-integrated circuit in an implantable device.

FIG. 15 shows vital sign response time during a medical emergency.

FIG. 16 is a table of sensor parameters.

FIG. 17 shows in vivo measurements of respiratory rate (RR) and heart rate (HR) from PPG, ECG, and IMU sensors, and measurements of temperature from a temperature sensor in an implanted device during an opioid overdose and respiratory recovery.

FIG. 18 shows in vivo measurements of HR from a microphone in an implanted device.

### DETAILED DESCRIPTION

Continuous monitoring of a subject's vital signs, including respiratory rate (RR), heart rate (HR), HR variability, galvanic skin response, skin temperature, blood oxygen saturation (SpO2), and blood pressure (BP), can provide early detection of biological events such as clinical deterioration in various disease states. Early detection of abnormal vital signs may herald worsening clinical disease. Early detection can trigger medical interventions to mitigate the risk of morbidity and manage the course of disease. Several disease treatments, including opioid overdose, epilepsy, and hypoglycemia (e.g., from an insulin overdose), may directly benefit from continuous monitoring of vital signs.

An implantable, autonomous closed-loop device can detect a subject's medical event and provide treatment. The device includes a sensing module including a variety of sensors that monitor various vital signs. The sensing module may sense respiratory rate (RR), heart rate (HR), HR variability, galvanic skin response, skin temperature, blood oxygen saturation (SpO2), and/or blood pressure (BP) to detect a medical event. The sensors in the sensing module may include an inertial measurement unit (IMU), an electrocardiogram (ECG) sensor, a photoplethysmogram (PPG) sensor, a microphone, and/or a temperature sensor. The IMU may include one or more 1-, 2-, or 3-axis accelerometers, 1-, 2-, or 3-axis gyroscopes, and/or 1-, 2-, or 3-axis magnetometers. In place of or in addition to an IMU, the sensing module may include a separate accelerometer, gyroscope, and/or magnetometer. The sensing module includes a combination of sensors to increase the sensitivity and precision of the device, and substantially decrease the possibility of false-positive results. The measurements from the IMU, accelerometer, gyroscope, microphone, and/or magnetometer sensors may be used to process ECG and PPG measurements to cancel noise associated with the subject's body movements.

The device also includes a therapeutic module including a motorized pump, a drug reservoir, and an actuator to automate the delivery of life-saving medicine to the subject. The sensing module and the therapeutic module are communicatively coupled so that when the sensing module detects a medical event, the therapeutic module is automatically triggered to deliver medicine in response to the medical event. The device may include control circuitry (e.g., a microprocessor) that regulates signals between the sensing module and the therapeutic module. The control circuitry may be connected to a wireless communication component (e.g., a transmitter, receiver, transceiver, and/or antenna) to communicate with an external device. The device also includes a battery that provides power to the sensing module and the therapeutic module. The battery may be rechargeable and may be charged wirelessly using an inductive wireless charging component.

The device is configured to be implanted subcutaneously in a subject. The device may have a cylindrical shape, a rectangular prism shape, or another shape. The volume of the device is about 1 cm³ to about 7 cm³ (e.g., 1.3 cm³ or 6.2 cm³). In the case that the device is implanted via surgical implantation, the device may have any suitable shape. For example, the surgically implanted device may be a rectangular prism having dimensions of about 65 mm length by about 12 mm width by about 8 mm height. In the case that the device is implanted via an injection, the device has a cylindrical shape. For example, the injectable device may have dimensions of about 4.5 mm diameter by 80 mm length.

Previously, conventional wearable device technology suffered low long-term adherence because many subjects were not willing to wear the devices continuously and correctly. Subjects removed these conventional wearable devices to charge them, or during bathing or physical activity, which disrupted their function. Once a subject disengaged with a conventional wearable device, there was a risk that they would stop using the device Additionally, subjects may experience a stigma associated with wearing a device for certain conditions like substance use disorders. This stigma may decrease a subject's willingness to use the device.

The implantable, closed-loop device is fully implanted within the subject to eliminate issues related to wearable technology adherence. Because it is fully implanted, the subject cannot easily remove the device. The subject does not have to worry about removing the device when bathing or exercising, or when the device needs to be recharged. As a result, sensor monitoring is not disrupted by any of these activities. Additionally, there is no risk that a subject will forget to put the device back on after these activities, because the subject does not remove the device to engage in these activities. Furthermore, because the device is fully implanted, there is little to no obvious visible sign that the subject is using the device, and therefore little to no stigma associated with using the device. The device can operate independently of manual user input, so that the subject does not need to constantly monitor it.

To extend the device's life span, the drug reservoir can be refilled while implanted during standard clinical visits. The drug reservoir is made of a flexible material and has a high volumetric loading efficiency. The reservoir has a protruding septum that is used for refilling the reservoir via injection with a syringe and needle. For example, a clinician may use a 30-G syringe needle injection to refill the reservoir in the implanted device. The drug reservoir has two openings, one is connected to the drug delivery valve and the other one is connected to the refill septum.

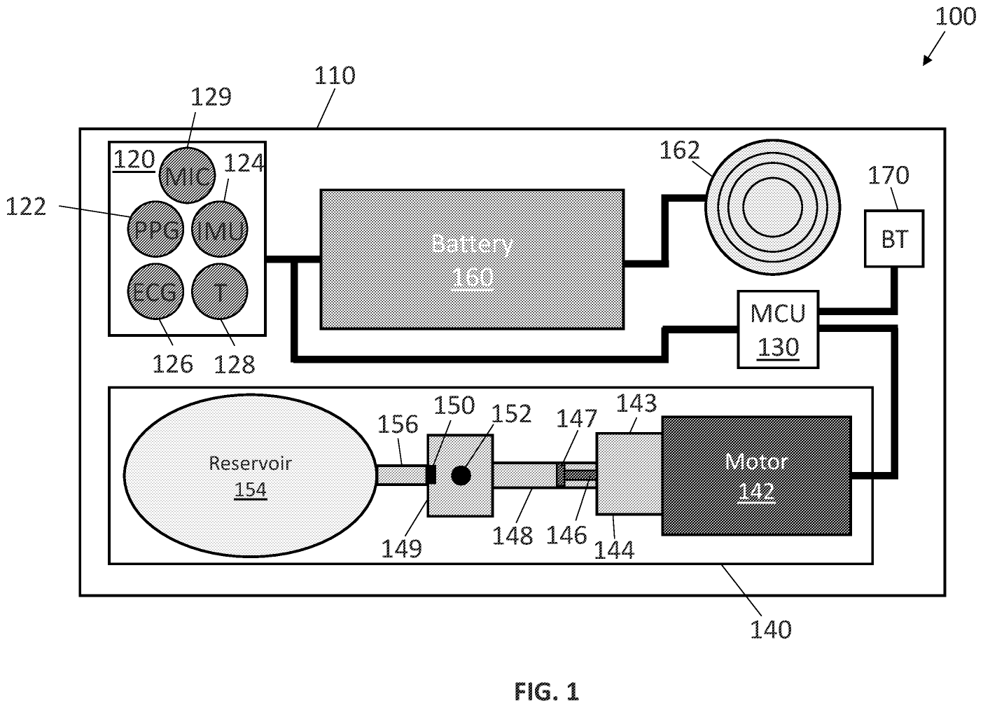

FIG. 1 shows an embodiment of a closed-loop implantable device 100. The device includes a housing 110. The housing provides a waterproof enclosure that houses and protects the device components. The housing is made of a biocompatible material and may be 3D printed (e.g., using a Stratsys printer). A sensing module 120 is disposed at least partially inside of the housing 110. The sensing module 120 includes a PPG sensor 122, an IMU sensor 124, an ECG sensor 126, a temperature sensor 128, and a microphone 129. Collectively, the sensors are used to detect a medical event in the subject. The IMU includes a 3-axis accelerometer, a 3-axis gyroscope, and a 3-axis magnetometer. The IMU measures body movements and can be used to measure respiratory rate and determine a state of consciousness (e.g., alert/awake, asleep, or unconscious). The ECG measures heart rate and respiratory rate and the PPG measures SpO2 and heart rate. The temperature sensor measures body temperature. There are at least two openings in the housing for ECG electrodes, so that the ECG electrodes are exposed to the environment. As discussed further below, these sensors may be operated concurrently, sequentially, and/or intermittently.

The sensing module 120 is communicatively coupled to a microcontroller 130. The microcontroller 130 is also communicatively coupled to a therapeutic module 140. The microcontroller 130 receives signals from the sensing module and determines the presence of a medical event. If a medical event is detected, the microcontroller actuates the therapeutic module 140 to deliver a medicine to the subject to respond to and treat the medical event. The microcontroller consumes less than 100 mW of power and has a footprint area that is less than 70 mm².

The therapeutic module 140 includes a motor 142, a reciprocating pump 143, and a drug reservoir 154. The motor may be brushed or brushless. For example, in a larger version of the device configured to be surgically implanted, the device uses a brushed motor. In a smaller version of the device configured to be implanted via injection, the device uses a brushless motor. The pump 143 includes an actuation mechanism 144 mechanically coupled to a piston rod 146 and piston 147. The piston 147 is disposed inside of piston sheath 148 which is fluidically coupled to valve 149. The valve 149 includes an inlet valve 150 and an outlet valve 152. The reservoir 154 holds about 0.5 mL to about 1.5 mL (e.g., about 1 mL) of a medicine. A conduit 156 is fluidically coupled to the reservoir 154 and the valve 149 to transport the medicine to valve 149 via the inlet valve 150. The motor 142 is mechanically coupled to the piston rod 146 to drive pump actuation. The motor 142 may be a DC motor. The pump 143 is a positive-displacement pump. The actuation mechanism 144 translates the unidirectional rotation of the motor 142 to a reciprocating lateral movement of the piston 147. The lateral movement of the piston 147 towards the motor creates a partial vacuum inside the pump 143 that drives medicine from the reservoir 154 into the valve 149. The inlet valve 150 is a one-way valve that substantially prevents the backflow of medicine from the valve 149 to the reservoir 154. The lateral movement of the piston 147 away from the motor 142 creates a higher pressure in the valve 149 that drives the medicine out of the outlet valve 152 and delivers the medicine to the subject. The outlet valve 152 is a one-way valve that substantially prevents liquid outside of the device from entering the valve 149.

A battery 160 provides power to the components inside of the device 100, including the sensing module 120, the microcontroller, and the therapeutic module 140. The battery 160 may be a rechargeable battery. For example, the battery 160 may be a pin-type lithium-ion battery with a nominal voltage of 3.8 V, a diameter of 3.65 mm, a height of 20 mm, and a maximum continuous discharging current of 30 mA. The battery is configured to deliver 450 medicine doses and can operate for at least one week on a single charge. The device 100 may include a wireless charging component 162 that charges the battery using an inductive power transfer. In this way, the battery 160 can be recharged without using external device components or removing the device 100 from the subject. As an example, the wireless charging component 162 may be a Qi wireless charging pad.

The device 100 may include a wireless communication chip 170 that transmits data from the device 100 to an external remote device (e.g., a computer or a smartphone). In some cases, the wireless communication chip 170 may receive wireless signals from the external device to control operation of the device 100. The communication chip 170 is operably coupled to the microcontroller 130. The communication chip 170 may include a transmitter, receiver, transceiver, and/or antenna. As an example, the wireless communication chip 170 may be a Bluetooth (BT) chip.

The amount of medicine delivered is selected based on the concentration of the medicine in the reservoir and the desired therapeutic dose. For example, the medicine may have a concentration of about 10 mg per mL and the therapeutic dose may be 2 mg of medicine, so that 0.2 mL of liquid is delivered to the subject by the therapeutic module. The microcontroller may record the volume of liquid delivered and the number of doses delivered to determine when the reservoir has little medicine left or is empty. When the microcontroller determines that the reservoir is emptying, it may send a wireless signal to an external device (e.g., a smartphone) via the communication chip 170 to inform the subject and/or a clinician that it is time to refill the reservoir.

The sensing module 120 detects an abnormal medical event. The abnormal medical event is a condition or the onset of a condition in the body of the subject for which the implantable device can provide a medical intervention by releasing a medicine to the subject. The medical event may be a life-threatening condition, including opioid overdose, epilepsy, or hypoglycemia. For example, the medical event may be an opioid overdose, for which therapeutic module 140 may release a dose of fentanyl to the subject as treatment. As another example, the medical event may be hypoglycemia, for which the therapeutic module 140 may release a dose of glucagon to the subject as treatment. As another example, the medical event may be a seizure (e.g., because of epilepsy).

The sensing module 120 includes more than one type of sensor to increase the accuracy of the detection of an abnormal medical event compared to the accuracy of a single type of sensor. The sensing module 120 includes a PPG sensor 122, an IMU sensor 124, an ECG sensor 126, a temperature sensor 128, and a microphone 129. Some of the sensors in the sensing module 120 measure the same vital signs as another sensor in the sensing module 120. In this way, these sensors directly corroborate the accuracy of each other. Other sensors in the sensing module 120 measure different vital signs. The implantable device does not determine the presence of a medical event until all the measured vital signs indicate the presence of the medical event. In this way, the sensors in the sensing module cross-validate one another.

For example, the sensing module may provide cross-validation of RR by means of IMU, ECG, and PPG sensors and HR by means of ECG and PPG sensors. This cross-validation of RR and HR significantly reduces the likelihood of false-positive medical event detection. The IMU may include a 3-axis accelerator, 3-axis gyroscope, and 3-axis magnetometer The IMU measures movement of the entire body while the ECG and PPG sensor only measure movement induced by respiration. In addition, ECG captures electrical signal variation while the PPG sensor capture optical signal variation.

The microphone 129 may also provide cross-validation of RR and HR. The microphone 129 may record acoustic signals inside of the body (e.g., sounds made by the heart, lungs, or intestines, and/or blood flow in arteries and veins). For example, the microphone 129 may act as an implantable stethoscope that can acoustically measure heart beats and/or respiration. The movement of the heart during a heartbeat is transformed into an acoustic signal by the microphone's tailored flexible diaphragm. In some cases, the microphone 129 may capture heart beats more accurately than chest-implanted ECG or PPG sensors because the microphone 129 does not pick up as many data artifacts from movement of the chest induced by respiration. The microphone 129 may be an analog microphone or a digital microphone using an I²S (also called inter-IC sound) interface standard, or a pulse width modulated (PWM) compressor. The type of microphone 129 may change its signal-to-noise ratio. Preferably, the microphone 129 uses an I²S interface standard, which provides a low signal-to-noise ratio.

Prior to the detection of an abnormal medical event, the device 100 may operate in a low-power mode, where some of the sensors are operated continuously at low sampling frequencies (e.g., about 10 Hz to 30 Hz for the IMU sensor 124 and about 0.01 Hz to about 1 Hz for the temperature sensor 128) and some sensors are turned on and off. For example, the IMU sensor 124 may operate continuously at a sampling frequency of about 10 Hz to 30 Hz, preferably about 20 Hz; and the temperature sensor 128 may operate continuously with a duty cycle of 100% at a sampling frequency of about 0.01 Hz to about 1 Hz, preferably about 0.05 Hz. The ECG sensor 126 may be turned on at regular intervals with a duty cycle of about 5% to about 15%, preferably about 10%, and a sampling frequency when turned on of about 40 Hz to about 60 Hz, preferably about 50 Hz. The PPG sensor 122 may also be turned on at regular intervals with a duty cycle of about 0.5% to about 2%, preferably about 1%, and a sampling frequency when turned on of about 40 Hz to about 60 Hz, preferably about 50 Hz.

The microcontroller 130 determines the onset of an abnormal medical event using information from all of the sensors in the sensing module 120. Once an abnormal medical event is detected, the microcontroller 130 switches the device 100 to a high-power mode, where all sensors are operated continuously with a duty cycle of 100% and a high sampling frequency of about 100 Hz. An overdose may be detected by the ECG, PPG, IMU, and temperature sensors indicating a decreased heart rate and respiratory rate, and a decrease in body temperature. A Because of the high accuracy of the sensing module 120, the implantable device 100 can be operated autonomously and independently of manual input from the subject or any other person. The high accuracy of the sensing module significantly decreases the risk of an unnecessary medicine delivery from the therapeutic module 140 to the subject. When the implantable device 100 detects a medical event and delivers a medicine, the microcontroller 130 may activate the wireless communication chip 170 to transmit to an external device data related to the detection of the medical event and medicine delivery.

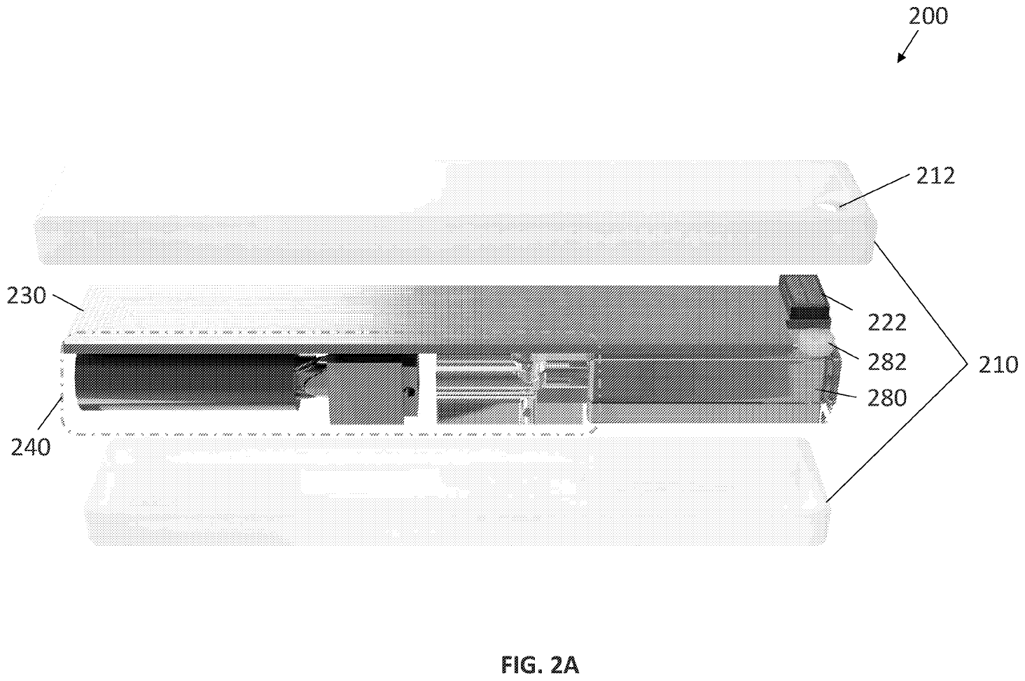

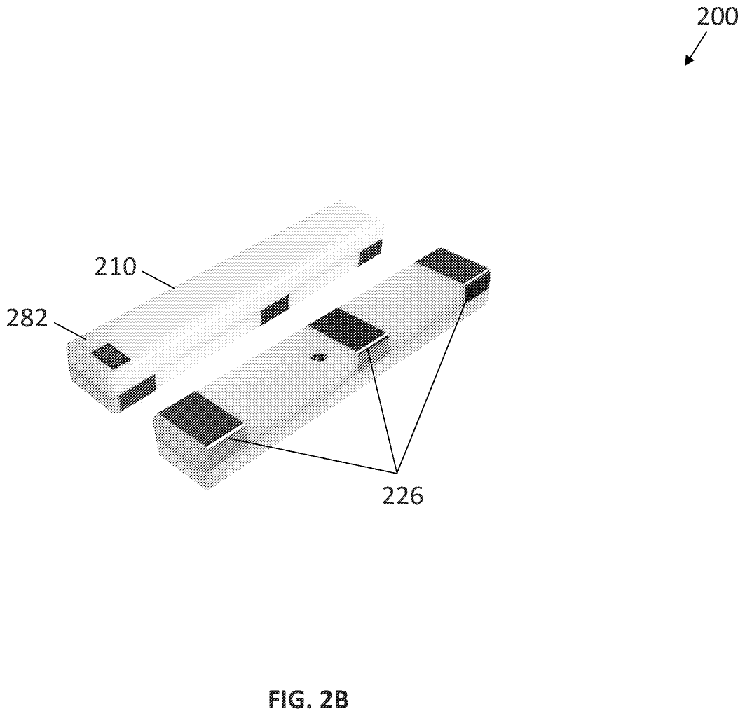

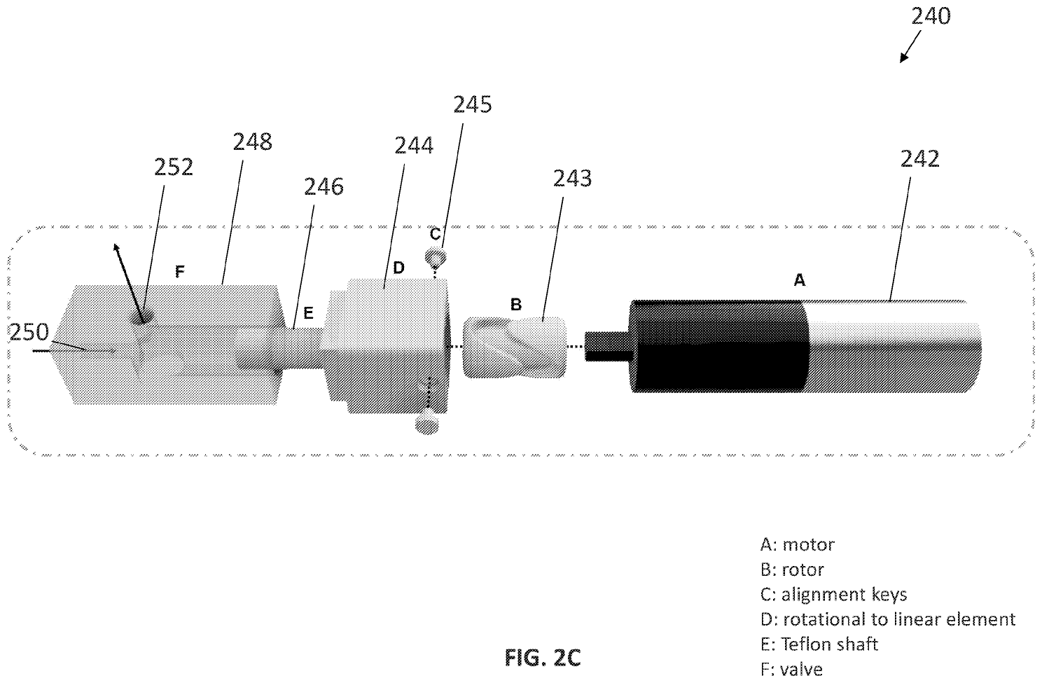

FIGS. 2A to 2C shows various views of another embodiment of a closed-loop implantable device 200. The device 200 includes a housing 210. The housing 210 is about 65 mm by about 12 mm by about 8 mm. The housing 210 provides a waterproof enclosure that houses and protects the device components. The housing 210 has an opening 212 for a septum 282 that is used to refill the medicine reservoir 280 via a needle and syringe. The reservoir 280 is fluidically coupled to the therapeutic module 240 in the implantable device 200. The housing 210 also has openings for the ECG electrodes 226 so that the ECG electrodes 226 are exposed to the environment around the device 200. The device 200 also includes a controller 230 on a printed circuit board. A wireless charging coil is also disposed on the printed circuit board. The wireless charging coil wireless recharges the battery in the device 200 that powers all of the components in the device 200. The device 200 also includes a PPG sensor 222.

FIG. 2C shows an exploded view of the therapeutic module 240 in device 200. The therapeutic module 240 includes a motor 242 and an actuation mechanism 244. The actuation mechanism 244 includes a rotational to linear element, a rotor 243 aligned with alignment keys 245. The actuation mechanism 244 is fluidically coupled to valve 248 via a shaft 246. The shaft 246 may be made of PTFE. The valve 248 includes an inlet 250 through which medicine enters the valve 248 from the reservoir 280, and an outlet 252 through which the medicine leaves the valve and is delivered to the subject.

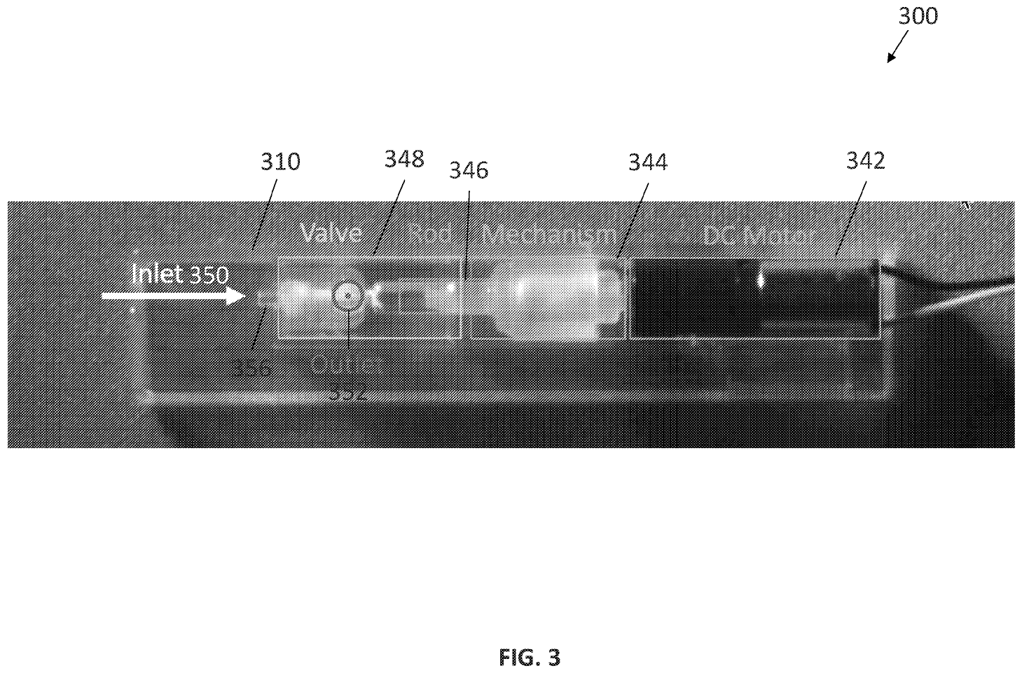

FIG. 3 shows a therapeutic module 300. The module 300 includes a housing 310 forming a cavity in which components are disposed. A DC motor 342 is disposed inside of the housing 310 cavity. The DC motor receives power from a rechargeable battery. An actuation mechanism 344 converts the rotational motion of the DC motor 342 to a lateral reciprocating pumping motion that moves rod 346 toward and away from the DC motor. When the rod 346 moves towards the DC motor, it creates a lower pressure in the valve 348, which draws medicine into the valve 348 through the inlet 350. When the rod 346 moves away from the DC motor, it creates a higher pressure in the valve 348 and drives the medicine out of the outlet 352. The module 300 can deliver medicine to the subject at a rate of about 5 μL per second to about 100 μL per second. For example, the module 300 can administer 500 μL of a liquid medicine in about 5 seconds.

### Energy Efficient Therapeutic Module Pump

One of the challenges in putting sensors and pump actuators in a single implantable device is power management. Generally, sensors and pump actuators have different power and energy requirements. Sensors may use low, continuous current draws from a battery for uninterrupted monitoring. Pump actuators may use high, short current draws from a battery for fast and pulsatile drug delivery. For example, each sensors may consume less than 1 mA of current, and a pump actuator may use about 30 mA to about 400 mA during operation. As an example, depending on the driving voltage, a pump actuator may have an inrush current greater than 100 mA over a duration of about 50 milliseconds (ms) and a steady state current draw of about 50 mA. To address this challenge, the implantable device may include a buffer circuit coupled to the battery and the microcontroller that drives actuators for a brief period.

Another challenge in putting sensors and actuators in a single implantable device is the battery's limited current draw. A battery's capacity and maximum current draw are proportional to its size. The smaller a battery is, the smaller the capacity and current draw. By its nature, an implantable device has a limited size, and therefore a limited battery size. Because of the battery's limited size, and because the battery is configured to power several different components with different power requirements, the device manages power carefully. One way that the device manages power carefully is by configuring the pump in the therapeutic module so that it has a limited current draw.

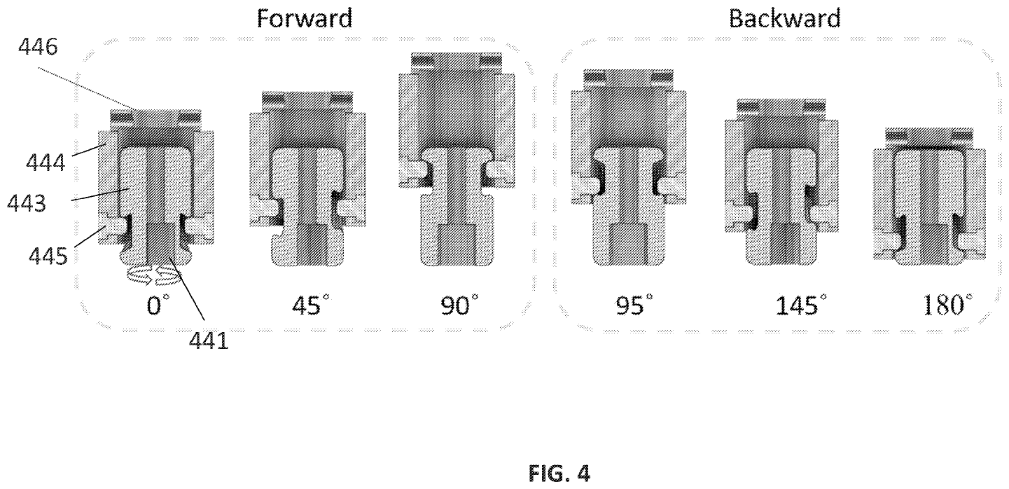

FIG. 4 shows the pump's actuation mechanism inside of the therapeutic module. The actuation mechanism converts the rotational motion of the motor into a lateral reciprocating motion. The DC motor moves in a unidirectional rotation and the actuation mechanism converts that movement to a bidirectional linear movement. The actuation mechanism includes a motor shaft connection 441 coupled to a rotor 443 held in place by alignment keys 445. The rotor 443 is inside of a rotational to linear element 444, which is fluidically coupled to a shaft 446 that acts as a conduit between the actuation mechanism and the valve through which medicine is dispensed. The actuation mechanism works when the DC motor moves in either a clockwise or counterclockwise direction. One revolution of the motor results in two reciprocating cycles. The pumping speed can be modulated by controlling the driving voltage to the DC motor. While the motor operates, the current draw by the motor is measured to serve as a feedback signal.

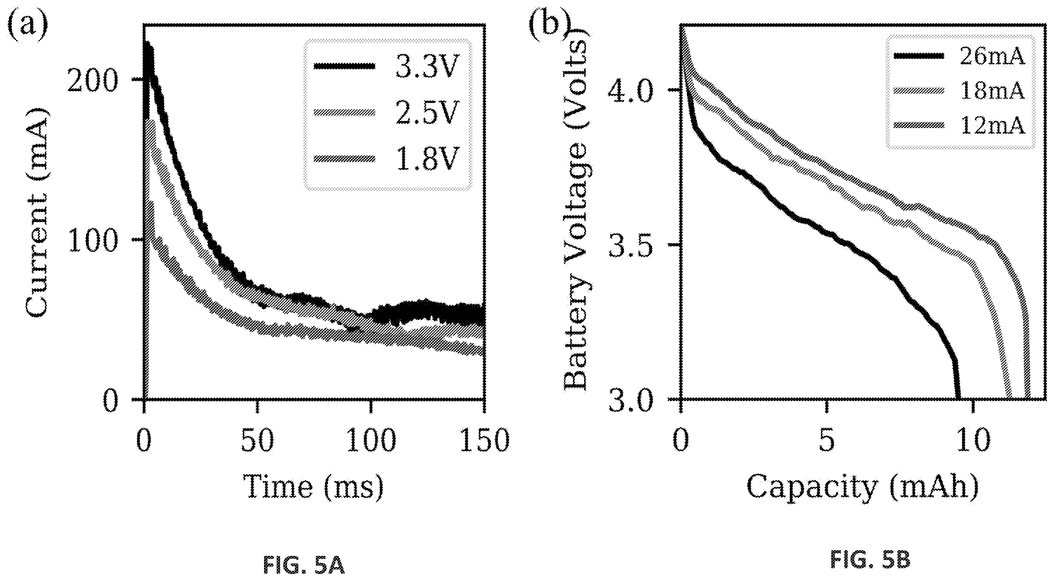

FIGS. 5A-10C show measurements of inrush current and voltage upon starting the pump motor shown in FIG. 2C using various start-up strategies. Generally, a motor suffers an inrush current when it is started. The inrush current may be two to ten times higher than the steady state current. Because of the inrush current, starting the pump motor when the therapeutic module is activated may be a significant power draw that may attenuate the battery lifetime. Most batteries are not configured to support the short, high current draw of the inrush current. The pump motor suffers more inrush current than a motor not attached to a pump, because the pump motor experiences friction from different mechanical components and liquid pressure. Therefore, the pump motor uses higher voltage and current to activate initial movement than the steady state driving voltage and current.

FIG. 5A shows current draw vs. time from the motor using various voltages. The inrush current draw induced by initially turning on an electrically powered actuator varies with driving voltage. The inrush current amplitude is proportional to the driving voltage and is typically five to ten times higher than the steady-state current.

FIG. 5B shows voltage vs. capacity from the using various currents. The discharge voltage profiles are characteristic of the battery. In one example, the battery can handle maximum continuous current draw of 30 mA and capacity of 15 mAh. Although the currents in FIG. 5B are far below the maximum rating, the battery's capacity is sharply attenuated near the ends of the voltage window.

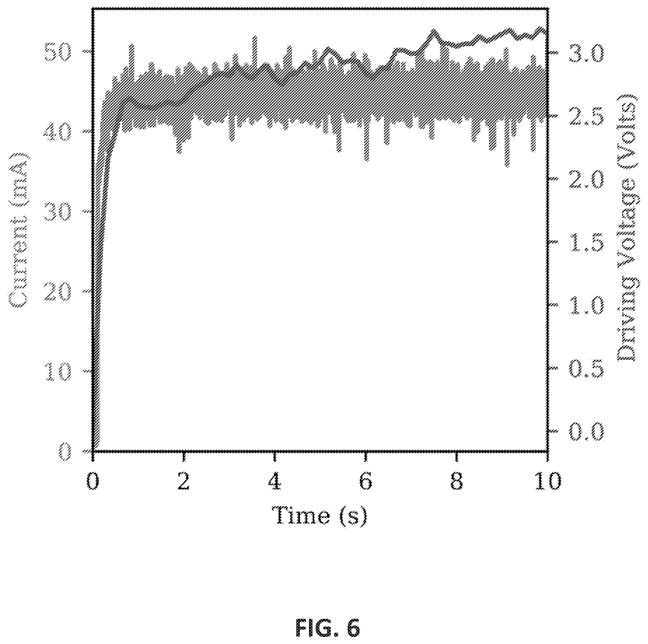

FIG. 6 shows inrush current draw vs. time for the motor using a current limiter. In the embodiment of the device used in FIG. 6, the circuit diagram includes a current limiter limiting the current drawn by the motor. A current limiter is one way to limit the inrush current. However, the current limiter itself consumes a significant amount of energy. The data in FIG. 6 shows that the current draw was limited to 50 mA or less using the current limiter, and no inrush current was induced. However, the motor took about 10 seconds to reach the desired driving voltage of 3.15 V. Therefore, this embodiment of the device is not well-suited for applications where faster actuation is desired.

A soft start method may be used to initiate pump motor movement while mitigating its inrush current. A soft start uses pulse-width modulation (PWM) that initially starts the motor movement at a lower speed and slowly increases the speed to a desired speed. Conventional PWM soft start methods create bundles of voltage pulses that create bundles of high current pulses, and place significantly higher loads on the battery.

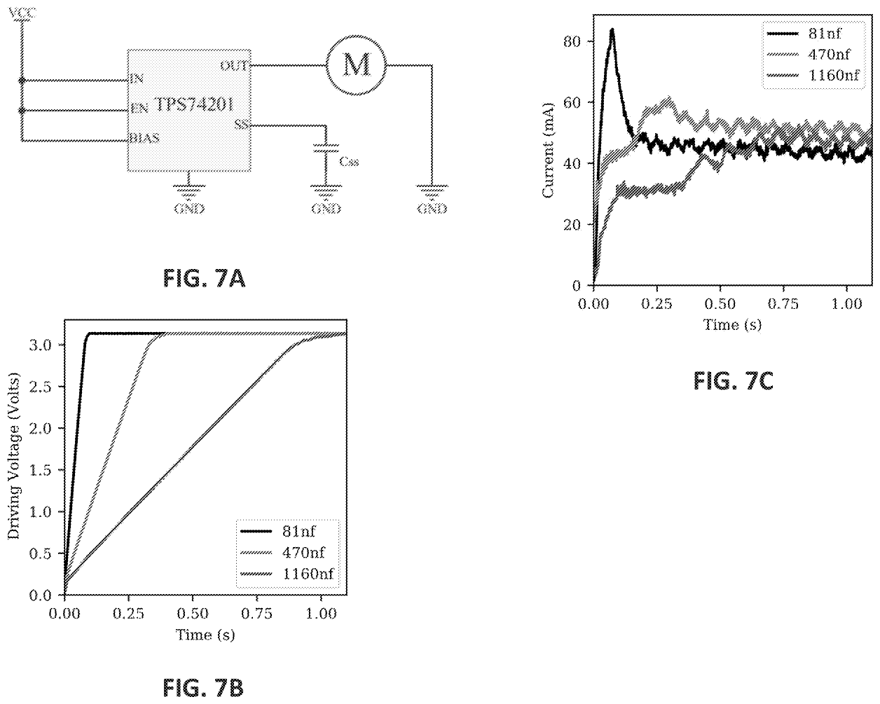

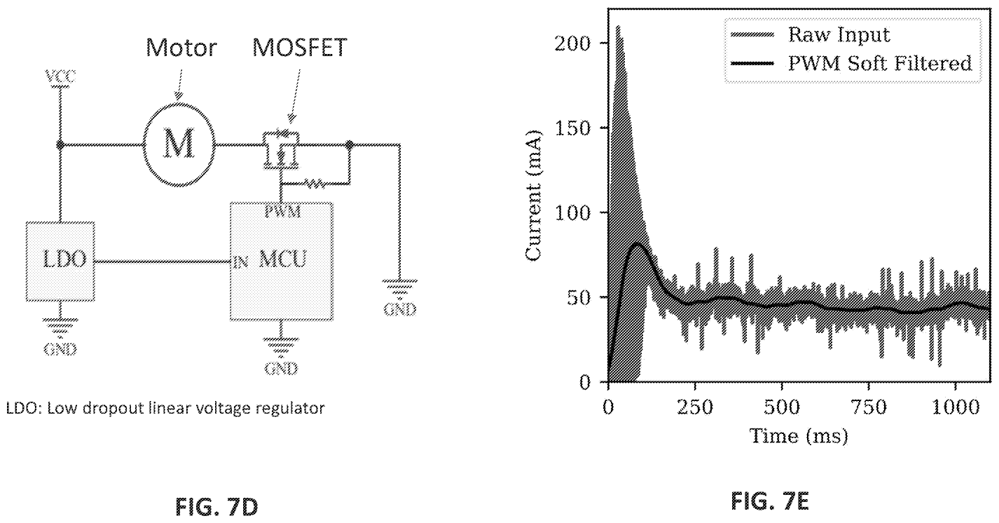

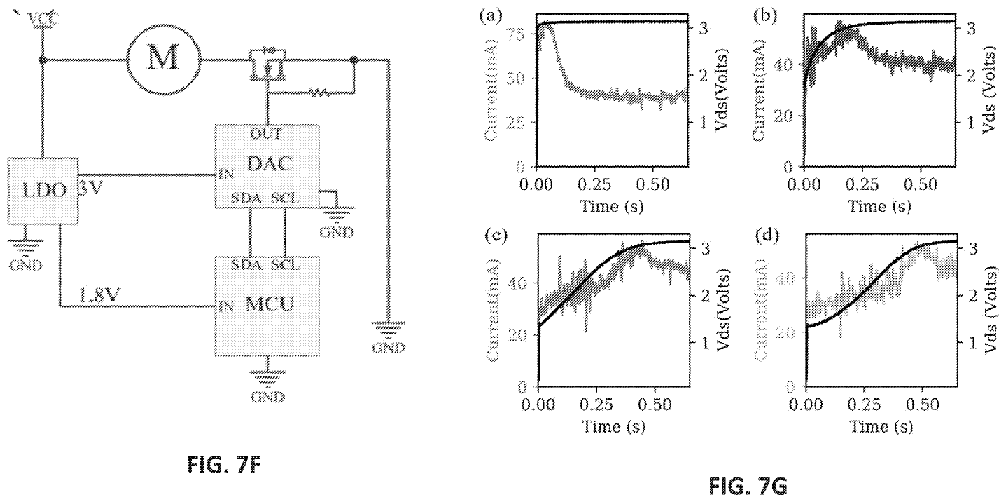

FIG. 7A shows a circuit diagram of a voltage regulator (TPS74201) used in one embodiment of the device to decrease the inrush current upon actuation of the motor using a soft start. The voltage regulator is part of a programmable soft starter. The voltage regulator uses a capacitor's charging characteristics to create a delay in the output voltage, which creates a soft start. The capacitance of the voltage regulator can be altered, with a higher capacitance voltage regulator having a longer charging period, and thus creating a slower soft start. FIG. 7B shows output voltage vs. time of the embodiment of the device with the voltage regulator shown in FIG. 7A. FIG. 7B shows different versions of the voltage regulator with various capacitance values. The data in FIG. 7B shows that the delay in the output voltage of the voltage regulator is proportional to its capacitance. FIG. 7C shows the current draw with varying the capacitances corresponding to the data shown in FIG. 7B. The slower the soft start, the lower the inrush current amplitude. Therefore, there is a trade-off between fast actuation and tempering the inrush current. The faster the soft start the higher the inrush current.

FIG. 7D shows a circuit diagram of components used for a pulse-width modulated (PWM) soft start of the motor in an embodiment of the device. The PWM components include a MOSFET, and a low dropout linear voltage regulator (LDO) electrically coupled to the microcontroller. The PWM-based soft start is controlled by the microcontroller. The microcontroller generated PWM signals and the MOSFET regulated the motor's speed. The PWM-based soft start slowly actuated the motor to decrease the inrush current. FIG. 7E shows inrush current vs. time of the motor using the PWM-based soft start. The black curve shows PWM soft filtered data of the inrush current using the PWM-based soft start. The grey curve shows the unfiltered data. The inrush current peak using this method is higher than that not using a soft start.

FIG. 7F shows a circuit diagram of components used for another PWM-modulated soft start of the motor in an embodiment of the device. In addition to the components used in the strategy in FIG. 7D, this version includes a digital-to-analog (DAC) converter to control the MOSFET and mitigate the trade-off between fast actuation and inrush current. FIG. 7G shows various inrush current and voltage draws from the motor using the PWM soft start strategy shown in FIG. 7F. The driving voltage reached the desired operational value quickly and the inrush current amplitude was attenuated from 160 mA to 80 mA or completely removed. The driving voltage reached the desired operational voltage in about 0.5 seconds or less.

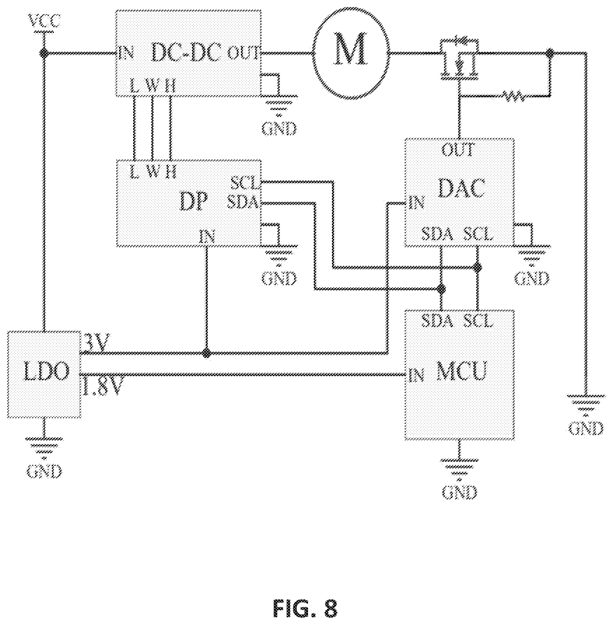

FIG. 8 shows a circuit diagram of a power management architecture in an embodiment of the device. This architecture reduces the inrush current and the continuous current draw from the battery.

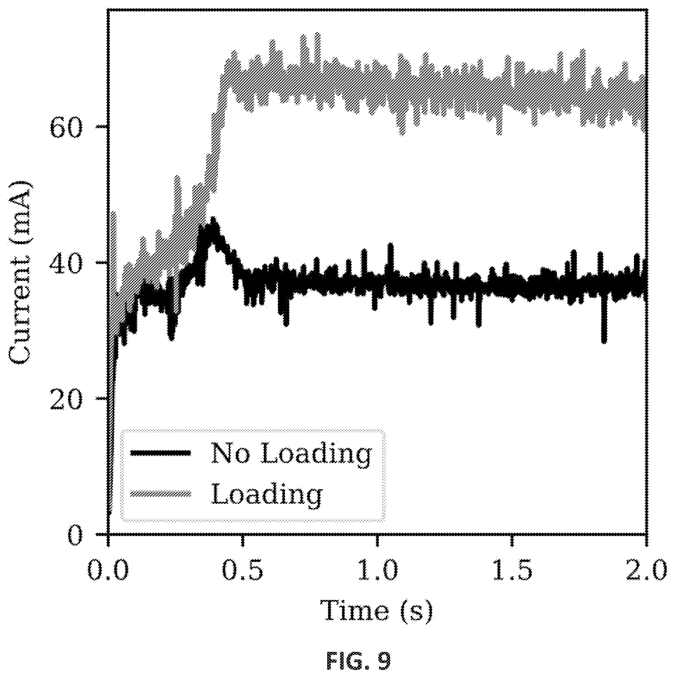

FIG. 9 shows current vs. time for a non-linear soft start of the motor under different loading conditions. The non-linear soft start method reduces inrush current and continuous current draw with and without a load. The driving voltage was regulated by means of the DC-DC converter. The pumping speed and current consumption are both linearly proportional to the driving. The energy consumption for delivering the same amount of medicine with various pumping speed depends on the driving voltage. The difference in energy consumption between the motor and battery is associate with the DC-DC converter efficiency loss. The energy consumption of the battery may be as low as 0.8 V. Using the DC-DC converter, the output current can be increased by lowering the output voltage without sacrificing the conversion efficiency.

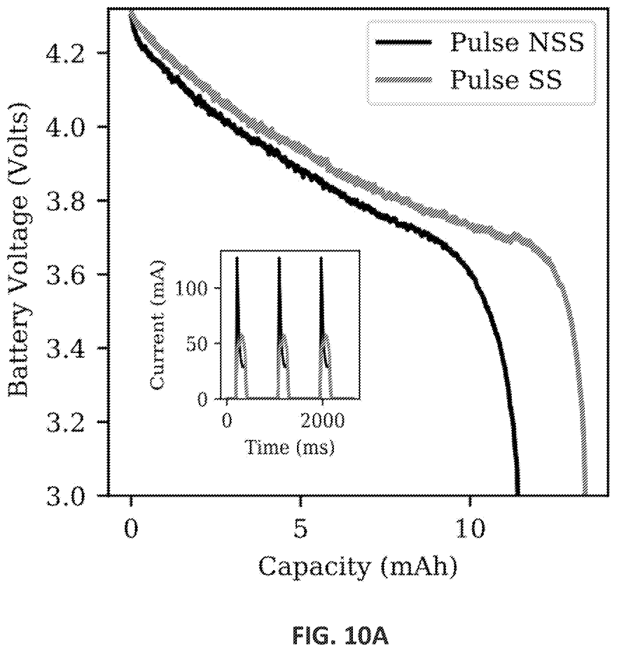

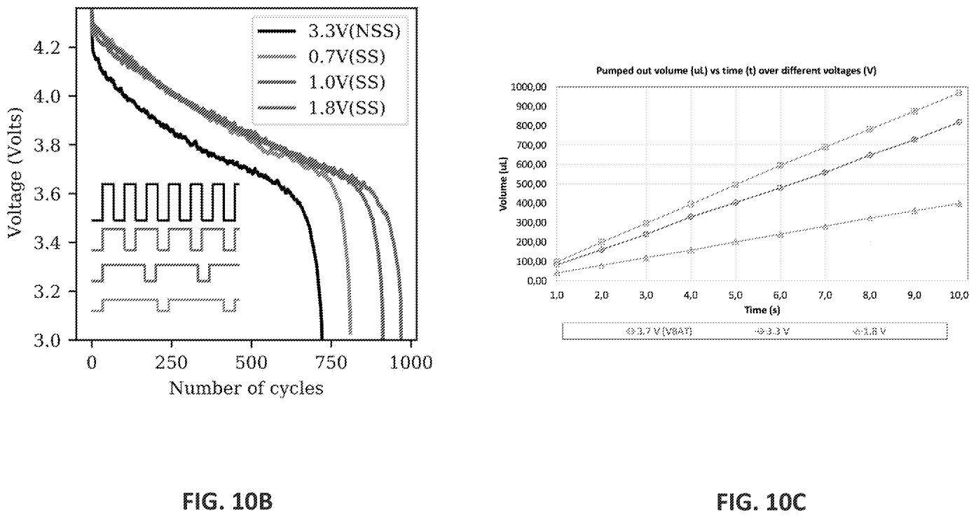

FIG. 10A shows battery voltage vs. capacity during PWM operation of the pump motor with a soft start (SS) and without a soft start (NSS). With a soft start, the battery maintains a higher capacity. Using a soft start to actuate the motor in the device may provide a longer battery lifetime, and therefore a longer device lifetime.

FIG. 10B shows voltage vs. number of cycles for pulsing a pump motor on and off using various soft start strategies. The various soft start strategies used different voltages and pulse widths. The total number of drug delivery cycles powered by the battery changes depending on the driving voltage. FIG. 10B shows the 1.8 V driving voltage resulted in the highest number cycles.

FIG. 10C shows pumped out volume of medicine vs. time using various driving voltages. The steady state current and the pumping rate were proportional to the driving voltage. The energy consumption for pumping a constant volume is a function of pumping time and driving voltage. The pump delivers medicine at a faster rate at a higher driving voltage.

The implantable device may include a closed-loop controller, such as a linear quadratic regulator (LQR) controller or a PID controller, to manage the energy consumption of the pump motor. The closed-loop controller monitors the real-time current draw and pump actuation frequency, the targeted output medicine volume, and the pumping period. The closed-loop controller receives feedback signals from a current sensor and a magnetometer to regulate the driving voltage of the pump. In this way, the closed-loop controller provides accuracy in the volume of drug administered and reduces total energy consumption.

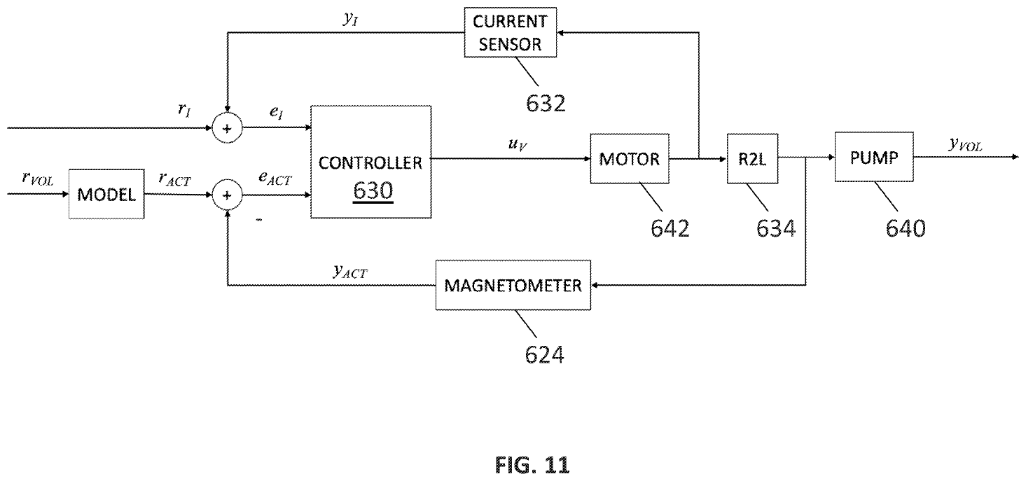

FIG. 11 is a simple circuit diagram showing connections between components in the closed-loop implantable device. The device includes a microcontroller 630 that provides a drive signal to the pump motor 642. The pump motor 642 drives the pump's actuation mechanism 634, which converts the pump motor's rotational motion to a reciprocating lateral motion, which dives the pump 640. A current sensor 632 is coupled to the microcontroller 630 and measures the current draw to the motor 642 to monitor energy consumption by the pump. A magnetometer 624 is part of the sensing module that is also coupled to the microcontroller 630.

### Implant Placement

The implantable device may be implanted into any of several locations within a subject's body. For example, the device can be implanted in the forearm, chest, or abdomen. The device may be implanted into a subject's subcutaneous tissue or transdermal tissue. Alternatively, the device may be implanted intraperitoneally. The device may be implanted with a horizontal orientation, a vertical orientation, or a diagonal orientation with respect to the subject's normal standing position.

The implantable device may be implanted into any living vertebrate animal. For example, the implantable device may be implanted into a mammal (e.g., a human, a pig, a dog, a cat, a cow, or a horse), a bird, a reptile, an amphibian, or a fish. Preferably, the implantable device is implanted into a human, a pet, a working animal, or livestock.

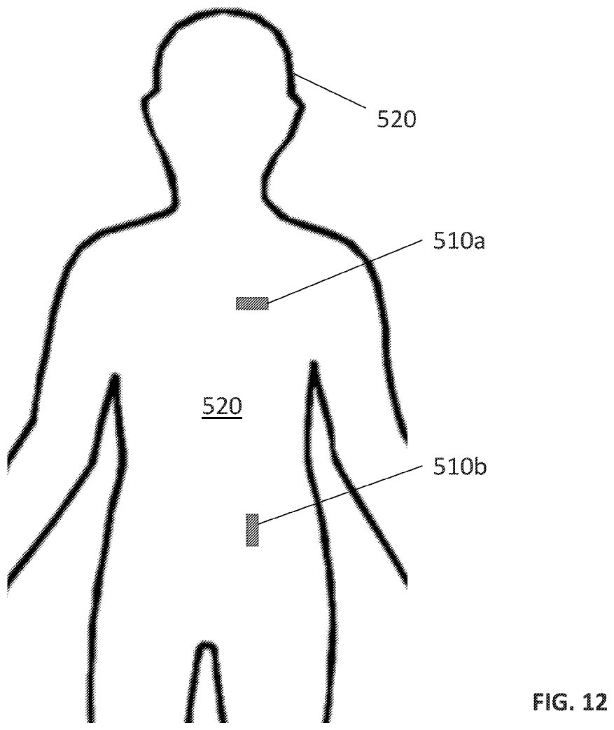

FIG. 12 shows two positions and orientations in which the implantable device may be placed in a human subject 520. Implantable device 510a is implanted in the chest of the human subject 520 with a horizontal orientation with respect to the subject's normal standing position. Alternatively, the implantable device 510a may be implanted in the chest of the human subject 520 with a vertical orientation with respect to the subject's normal standing position. Implantable device 510b is implanted in the abdomen of the human subject 520 with a vertical orientation with respect to the subject's normal standing position. Alternatively, the implantable device 510b may be implanted in the abdomen of the human subject 520 with a horizontal orientation with respect to the subject's normal standing position. The abdomen may be a preferred location for implantation of the implantable device because the adipose tissue and subcutaneous tissue may more easily accommodate a device with a larger volume. The abdomen may also be a preferred location for administration of a medicine by the implantable device. Alternatively, the implantable device may be implanted into a limb (e.g., arm or leg) of the subject. The position and orientation of the implantable device affects the performance of the sensing module in the implantable device.

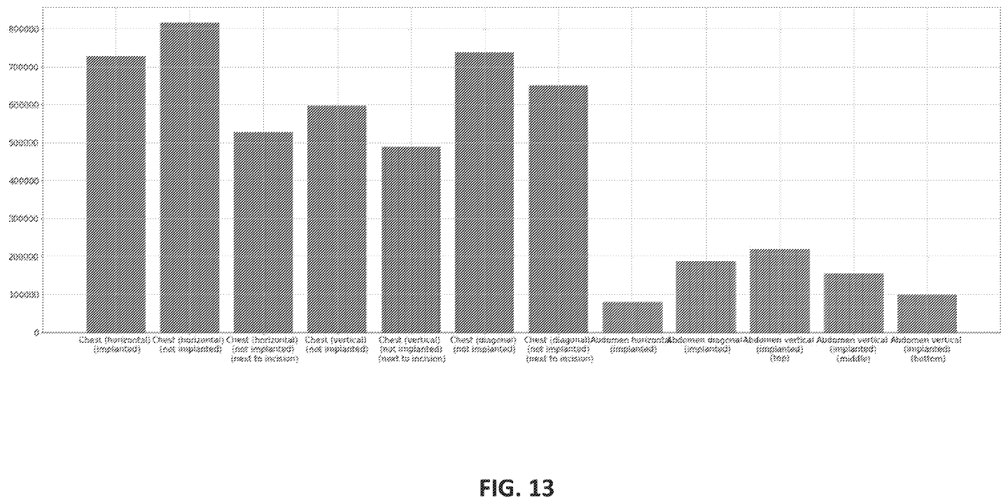

FIG. 13 shows a graph of ECG signals measured by ECG sensors placed in various positions and orientations inside of a swine model. The height of the bars indicates the strength of the ECG signal. The seven bars on the left of the graph show ECG sensors positioned at the chest. The five bars to the right of the graph show ECG sensors positioned at the abdomen. The ECG sensors at the chest measured stronger heart rate and respiratory rate signals. However, the signals from the ECG sensors positioned at the abdomen had better signal-to-noise ratios than those positioned at the chest. Therefore, it is possible to position the implantable device in the chest or the abdomen.

FIG. 13 also shows signal dependence on the orientation of the ECG sensor. The ECG sensor was placed horizontally, vertically, or diagonally with respect to the swine model's normal standing position. For comparison, control ECG sensors were not implanted and instead placed on top of the skin. In the abdomen, diagonally-oriented and vertically-oriented ECG sensors measured higher signals than horizontally-oriented ECG sensors. Implantation at the top of the abdomen (i.e., closer to the heart) resulted in a higher signal than implantation at the bottom of the abdomen. Overall, there was no significant difference in the ECG signal intensity from the device implanted with various orientations in the chest or abdomen of the subject.

### Sensing Module Duty Cycling

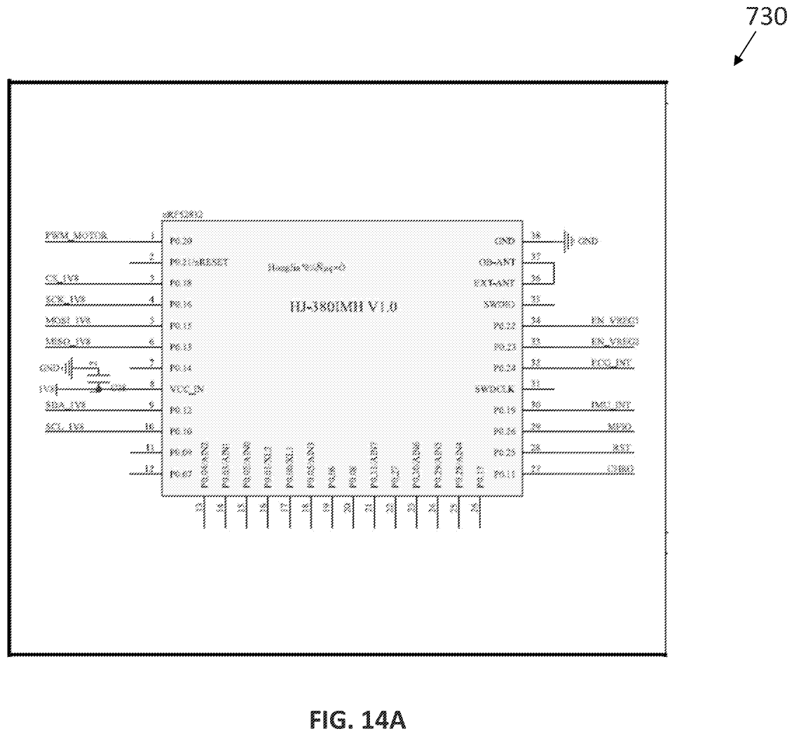

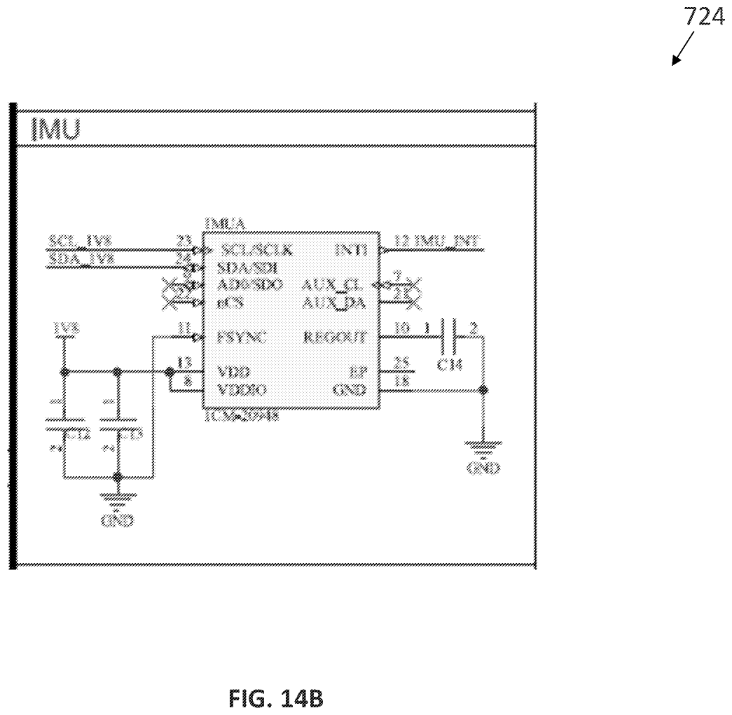

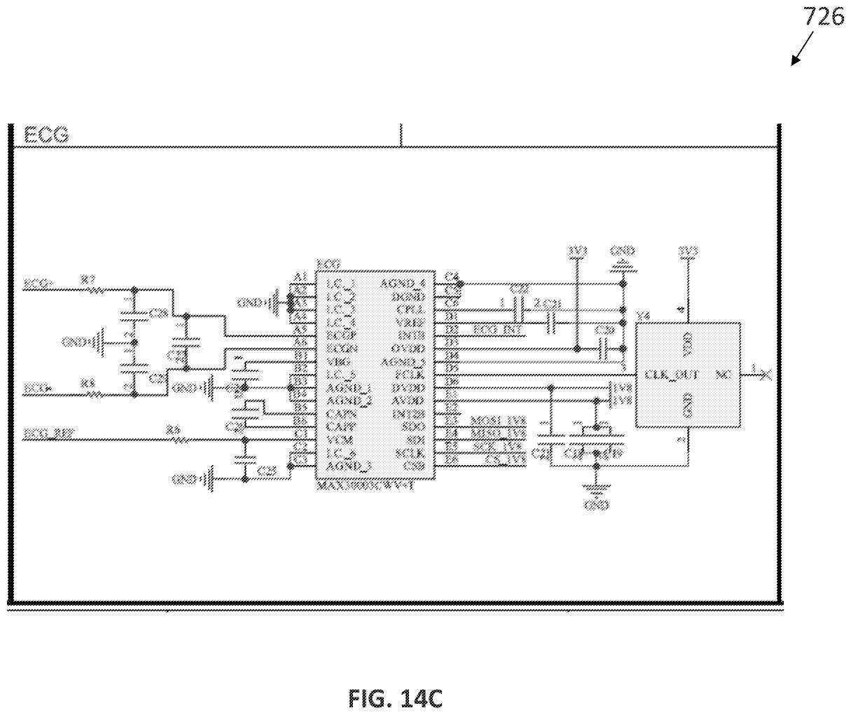

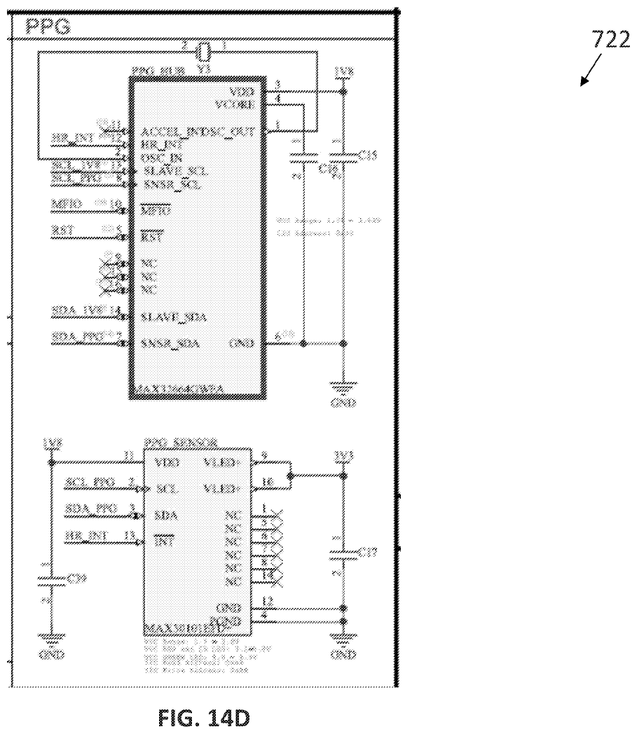

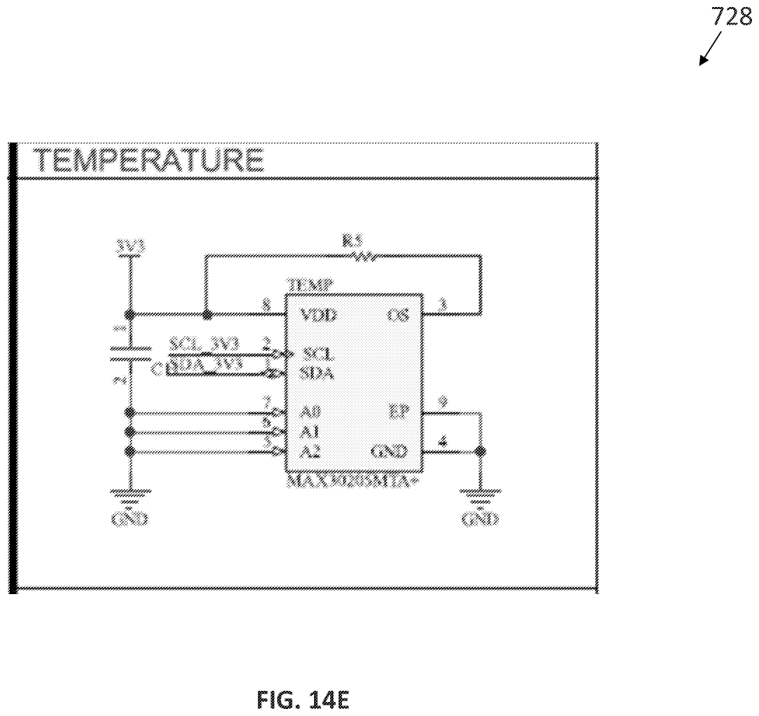

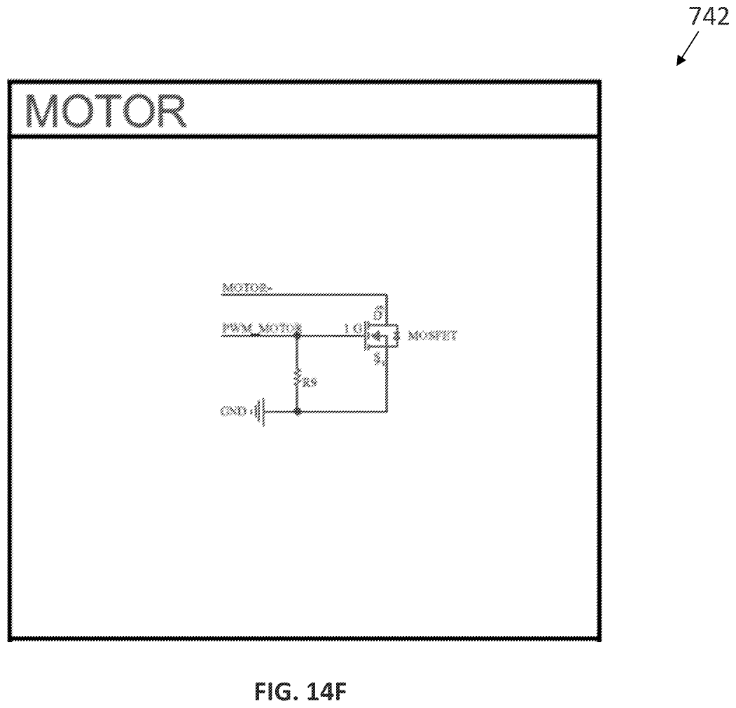

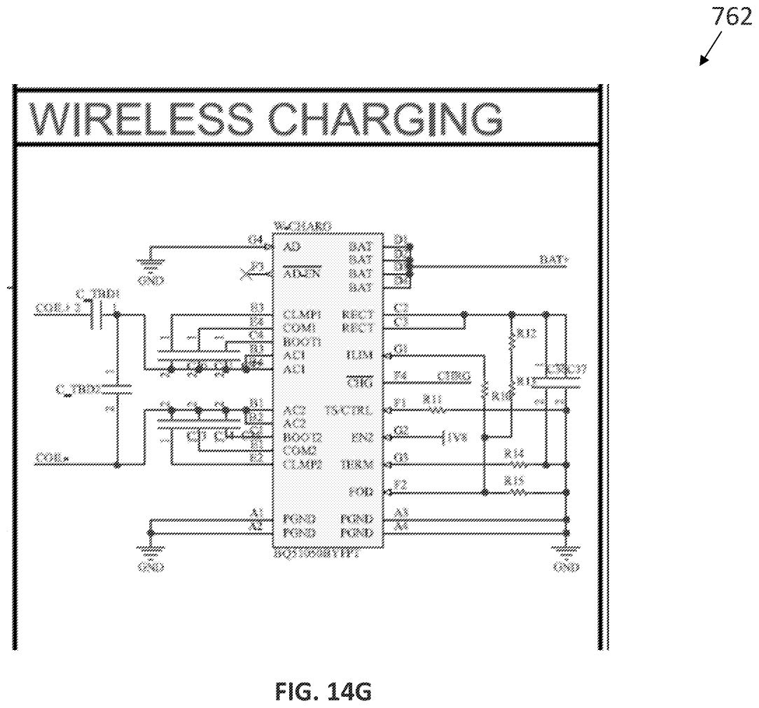

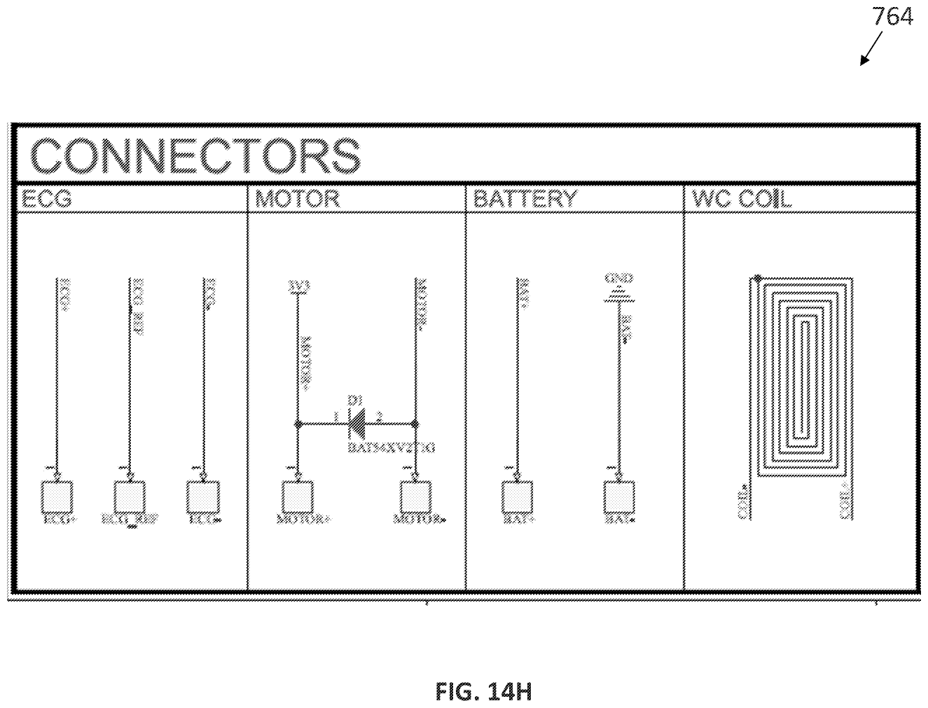

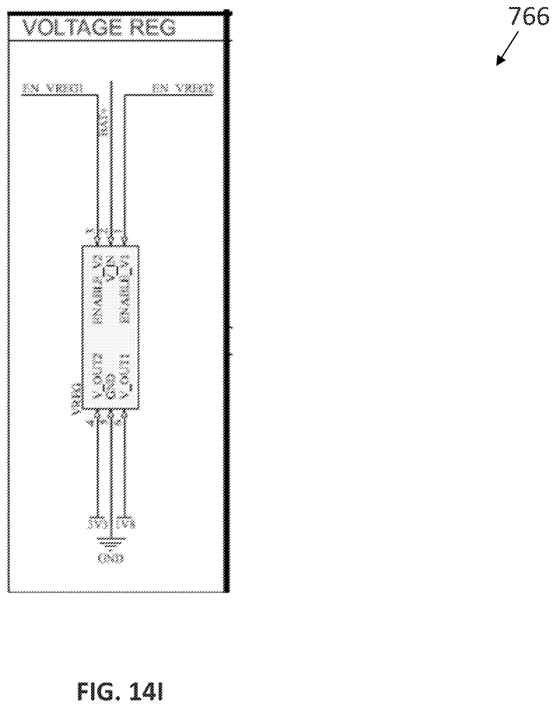

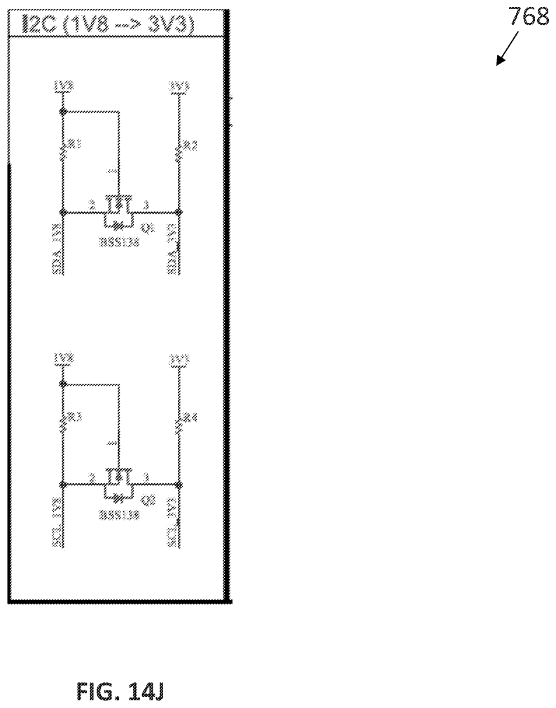

FIG. 14A-14J are circuit diagrams showing electrical connections between the microcontroller and several of the components in the implantable device. FIG. 14A shows the circuit diagram 730 for the microcontroller. The microcontroller is connected to the IMU sensor (whose circuit diagram 724 is shown in FIG. 14B), the ECG sensor (whose circuit diagram 726 is shown in FIG. 14C), the PPG sensor (whose circuit diagram 722 is shown in FIG. 14D), the temperature sensor (whose circuit diagram is shown in FIG. 14E), the pump motor (whose circuit diagram 742 is shown in FIG. 14F), and the wireless charger (whose circuit diagram 762 is shown in FIG. 14G). The components are connected via connectors, as shown in circuit diagram 764 in FIG. 14H. The system also includes a voltage regulator, whose circuit diagram 766 is shown in FIG. 14I, that regulates the voltage to provide two different reference voltages to the microcontroller and sensors, respectively. The system also includes an inter-integrated circuit (i.e., I2C), whose circuit diagram 768 is shown in FIG. 14J. The inter-integrated circuity is a wired communication protocol to interface with digital sensors, including the temperature sensor, IMU sensor, and PPG sensor. Alternatively, an SPI is another type of wired communication protocol that may be used in place of the I²C with a higher data rate to interface with the ECG. In an embodiment, the microcontroller, Bluetooth chip, and antenna are all integrated in a single chip to reduce the overall circuit footprint area.

The microcontroller may implement embedded deep learning in the implantable device. Deep learning is used to implement more complicated sensor fusion algorithms. Moreover, the microcontroller may engage in deep learning by itself without transmitting the raw data from the sensors to an external device, which in turn may reduce energy consumption.

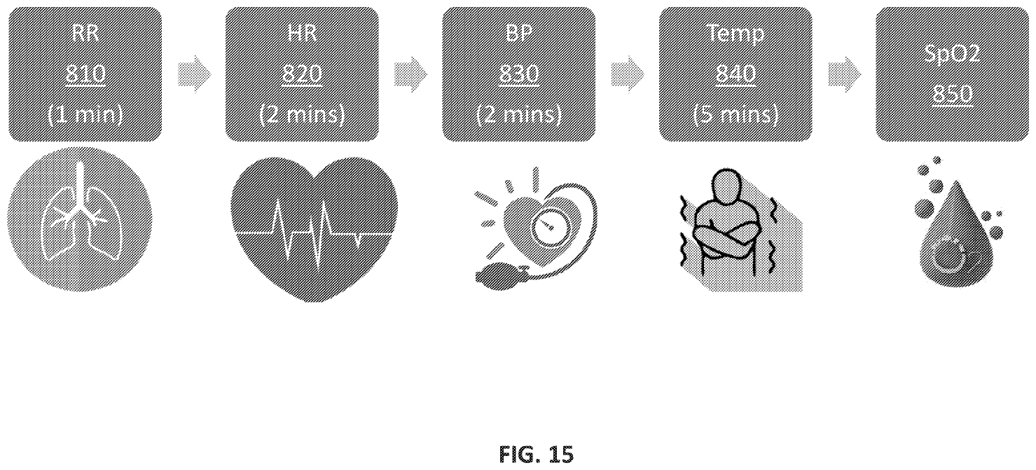

FIG. 15 shows the response times of different vital signs of a subject experiencing an opioid overdose. FIG. 15 shows that, upon the onset of an opioid overdose, a change in the respiratory rate (RR) 810 occurs within 1 minute, a change in heart rate (HR) 820 occurs within 2 minutes, a change in blood pressure (BP) 830 occurs within 2 minutes, a change in body temperature (temp) 830 occurs within 5 minutes, and a change in blood oxygen saturation (SpO2) 850 also occurs. In the example of opioid overdose, death is preceded by respiratory depression resulting in hypoxia and subsequent bradycardia. Clinical observations show that decrease of respiratory rate (RR) is first observed, followed by the decrease of heart rate (HR), and then hypoxia. Opioid delivery induces a body temperature elevation of about 2° C. In contrast, the delivery of Naloxone decreases the body temperature about 0.2° C. in about 5 minutes to 10 minutes.

Since the series of vital sign changes upon the onset of a medical event (e.g., opioid overdose) are predictable, the implantable device can use these vital sign changes to determine the onset of the medical event. For example, the vital sign changes upon opioid overdose are distinct from vital sign changes that occur during normal life and other medical conditions. Because the vital sign changes are distinct, these changes can be used to differentiate an opioid overdose from other etiologies of respiratory depression (e.g., normal sleep and sleep apnea) from opioid overdose events.

In an embodiment, the implantable device does not continuously and simultaneously measure vital signs from all the sensors in the sensing module. Instead, sensors are duty cycled and each sensor has a different sampling rate. The sampling rate of a sensor may depend on a vital sign response time to a medical event measured by the sensor. The sampling rate of a sensor may also depend on its power consumption. In this way, some sensors operate more frequently than others. By cycling operation of some sensors with lower sampling rates, the sensing module conserves power so that the implantable device can operate for longer periods of time before its battery needs to be recharged.

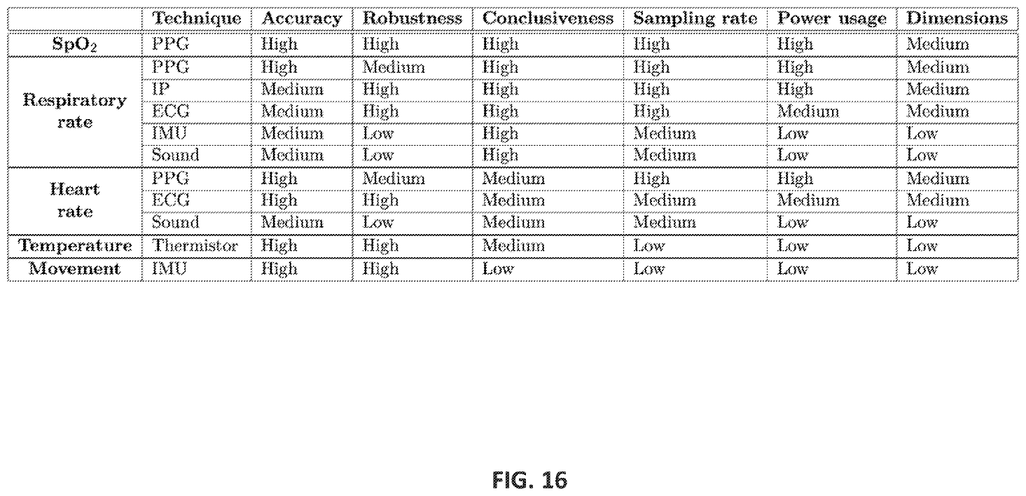

FIG. 16 is a table of sensor parameters. The table shows that SpO₂ can be measured by PPG. Respiratory rate can be measured with PPG, impedance pneumography (IP), ECG, IMU, and acoustic sound sensors. Heart rate can be measured with PPG, ECG, and sound sensors. Temperature can be measured with a temperature sensor (e.g., a thermistor or a thermocouple). Body movement can be measured with an IMU sensor. The table also gives accuracy, robustness, conclusiveness, sampling rate, power usage, and dimensions of each of these sensors. Blood pressure (BP) may be determined from the measurement of pulse signal from ECG and PPG sensors by means of pulse transient time (PPT).

As an example, the IMU sensor may be operated with the highest duty cycle. The IMU sensor may monitor the subject's body movement to detect consciousness and respiration. If the IMU senses any abnormality in consciousness or respiration, the microcontroller may trigger the ECG sensor to measure the RR and the HR. If the results of IMU and ECG sensors are consistent, the microcontroller may trigger the PPG sensor to measure the SpO2. Meanwhile, body temperature may be measured every minute. If the RR is below 10 bpm, HR is below 60 bpm, body temperature is above 38° C., and the SpO2 is below 90%, the microcontroller may automatically activate the pump motor to deliver Naloxone from the drug reservoir. The implantable device may communicate with the subject's cellphone via Bluetooth with encryption and may trigger the subject's cellphone to contact or send a message to an emergency first responder, an emergency contact, or a hospital.

### Cyber Security

The closed-loop system is less likely to be subject to a cyber-attack. Since conventional wearable/implantable systems like continuous glucose monitors use wireless communication between an implanted sensor, an external drug delivery device, and the patient, there are opportunities where data input and transmission can be accessed and manipulated to trigger unnecessary and potentially dangerous drug administration. Because the implantable device is a closed-loop autonomous system, it does not need a continuous connection with other devices and is therefore less likely to be subject to a cyber-attack.

An encryption unit may be included in the embedded software of the control circuitry of the implantable device. The encryption unit may encrypt data transmitted to a remote device. Only a party with the encryption key can decrypt the data. Therefore, any external adversary is not able to decipher the data being transmitted. The encryption unit may use counter-based encryption to prevent replay attacks by external adversaries. A replay attack is a form of network attack in which valid data transmission is maliciously or fraudulently repeated or delayed. This is carried out either by the originator or by an adversary who intercepts the data and re-transmits it, possibly as part of a spoofing attack.

### Example Use of an Implantable Device to Detect and Treat Opioid Overdose

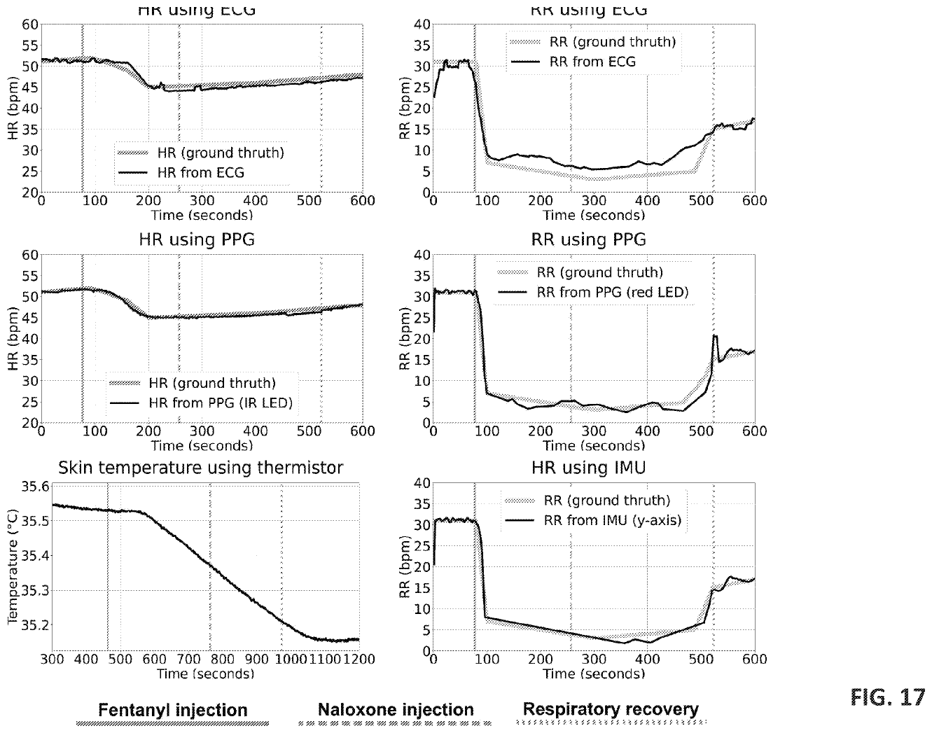

FIG. 17 shows in vivo vital sign measurements from an implantable device implanted subcutaneously in the abdomen of a pig model. The results show vital sign measurements from the pig model prior to and after a Fentanyl overdose and Naloxone delivery. FIG. 17 shows respiratory rate measurements as measured by PPG, IMU, and ECG sensors in the implantable device; hear rate measured by PPG and ECG sensors in the implantable device; and body temperature as measured by a temperature sensor in the implantable device.

Upon administration of the Fentanyl injection subcutaneously to the pig model, the respiratory rate declined rapidly. In contrast, cardiac activity (HR) continued for about 1 minute after administration of the Fentanyl injection before decreasing. After the decline of cardiac activity, hypoxia in the pig model ensued. About 180 seconds after Fentanyl administration, Naloxone was administered subcutaneously to the pig model and breathing resumed around 220 seconds after Naloxone administration.

FIG. 17 shows the RR variation measured by the accelerometer of the IMU sensor, the ECG sensor, and the PPG sensor prior to and after Fentanyl and Naloxone delivery. There was a dramatic drop in RR about 16 seconds after Fentanyl administration. Respiration typically stops within about 37 seconds after Fentanyl administration. The sensor measured a RR between 5 bpm and 10 bpm due to staff artificially bagging the pig to keep the animal alive.

FIG. 17 also shows HR variation measured by ECG and PPG sensors. HR started to decline about 26 seconds after Fentanyl administration. HR reached its lowest value about 244 seconds after starting to decline. HR reached a 10% drop about 78 seconds after Fentanyl administration. After starting to decline, the HR reached a 10% drop about 52 seconds later.

FIG. 17 also shows the temperature variation measured at the abdomen subcutaneously. The subcutaneous temperature gradually decreased after the Fentanyl administration. There was a drop of about 0.2° C. within 5 mins of Fentanyl administration. The temperature started to recover after the delivery of Naloxone. It took about 52 seconds from the administration of Fentanyl for the temperature to start dropping. It took about 520 seconds from the Fentanyl administration for the temperature to reach its lowest value.

### Example Use of an Implantable Device with a Microphone

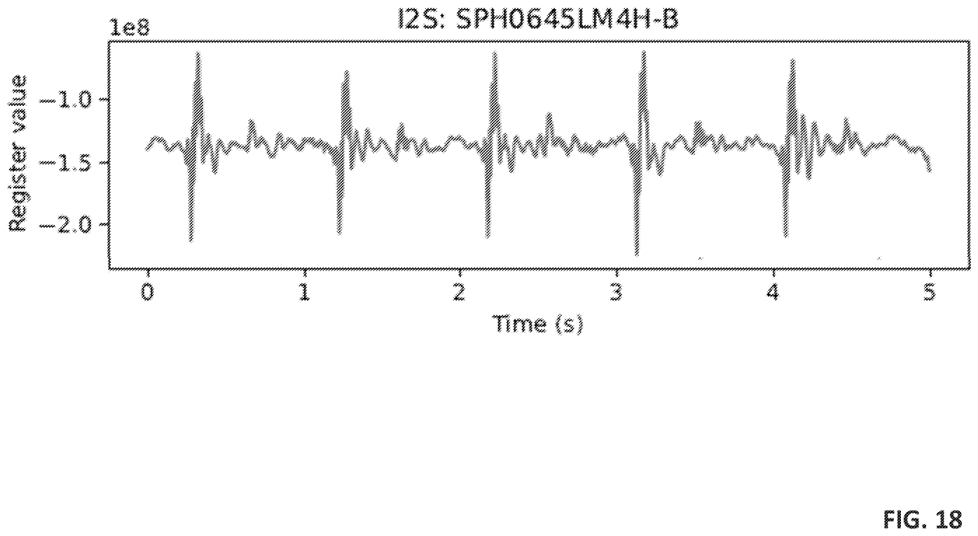

FIG. 18 shows in vivo measurements of HR from a microphone in an implanted device. For example, the microphone may be an Adafruit I²S microelectromechanical (MEMS) microphone breakout, which is incorporated in an implantable device implanted subcutaneously in the chest of a pig model. The microphone accurately measures acoustic signals from the heart without data artifacts due to motion of the chest during respiration, which can be present in data from ECG and PPG sensors implanted in the chest.

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A device configured to be implanted in a subject, the device comprising:
a sensing module comprising at least two of:
an accelerometer;
an electrocardiogram (ECG) sensor;
a photoplethysmogram (PPG) sensor;
a temperature sensor; and
a microphone;
a therapeutic module coupled to the sensing module, the therapeutic module comprising:
a drug reservoir; and
a reciprocating pump; and
a battery coupled to the sensing module and the therapeutic module, configured to provide power to the sensing module and the therapeutic module;
wherein:
the sensing module is configured to detect a biological event in the subject and, upon detection of the biological event, send a signal to the therapeutic module; and
the therapeutic module is configured to receive the signal from the sensing module and, upon receiving the signal, administer a drug to the subject from the drug reservoir via the reciprocating pump.

2. The device of claim 1, further comprising a controller operably coupled to the sensing module and the therapeutic module, the controller comprising at least one of:
a digital-to-analog (DAC) converter coupled to a switch; or
a direct current (DC) to DC converter.

3. The device of claim 2, further comprising a housing comprising a cavity; wherein the sensing module, therapeutic module, battery, and controller are disposed in the cavity.

4. The device of claim 1, wherein the accelerometer is part of an inertial measurement (IMU) sensor comprising:
a gyroscope; and
a magnetometer.

5. The device of claim 4, wherein the sensing module comprises:
the inertial measurement (IMU) sensor;
the electrocardiogram (ECG) sensor;
the photoplethysmogram (PPG) sensor; and
the temperature sensor.

6. The device of claim 1, wherein the reciprocating pump comprises:
a motor having a unidirectional rotation; and
a reciprocating actuation mechanism.

7. The device of claim 1, further comprising:
a wireless charging pad electrically coupled to the battery;
wherein the battery is rechargeable.

8. The device of claim 1, further comprising a wireless communications chip.

9. The device of claim 1, wherein:
the biological event is an opioid overdose; and
the drug is naloxone.

10. The device of claim 1, wherein:
the biological event is hypoglycemia; and
the drug is glucagon.

11. The device of claim 1, wherein the device is configured to be fully implanted in the subject's abdomen.

12. The device of claim 1, wherein the device is configured to be fully implanted in the subject's chest.

13. A method of using a device;
the device comprising:
a sensing module;
a therapeutic module comprising:
a drug reservoir containing a drug; and
a pump; and
a rechargeable battery configured to provide power to the device;
wherein the device is fully implanted in a subject;
the method comprising:
measuring the subject's respiratory rate, heart rate, blood oxygen saturation, and temperature with the sensing module;
while measuring, detecting a biological event in the subject; and
upon detecting the biological event, actuating a pump to administer the drug from the drug reservoir to the subject.

14. The method of claim 13, wherein actuating the pump comprises modulating a current pulse to the pump.

15. The method of claim 14, wherein modulating the current pulse comprises:
converting a control signal from a digital signal to an analog signal; and
controlling a switch with the analog signal;
wherein the switch controls the current pulse to the pump.

16. The method of claim 13, wherein actuating the pump comprises administering to the subject about 500 μL of the drug in about 5 seconds.

17. The method of claim 13, further comprising recharging the rechargeable battery via inductive coupling.

18. The method of claim 13, further comprising refilling the drug reservoir with the drug.

19. The method of claim 13, further comprising, upon detecting the biological event, sending a wireless signal to an external device alerting the external device about the biological event.

20. A system configured to be implanted in a subject, the system comprising:
a housing comprising a cavity;
a sensing module disposed in the cavity, the sensing module comprising at least two of:
a respiratory rate sensor;
a heart rate sensor,
a blood oxygen saturation sensor,
a temperature sensor; and
a microphone
a reciprocating pump coupled to the sensing module and disposed at least partially in the cavity, the reciprocating pump configured to administer a medicine to the subject; and
a battery disposed in the cavity and coupled to the sensing module and the reciprocating pump, the battery being configured to provide power to the sensing module and the reciprocating pump.
